(19) United States
(12) Patent Application Publication
(10) Pub. No.: US 2022/0133283 A1
(43) Pub. Date: May 5, 2022

(54) SWAB

(71) Applicant: The Aga Khan University, Karachi (PK)

(72) Inventors: Saleem Sayani, Wynnewood, PA (US); Muhammad Abdul Muqeet, Karachi (PK); Zahra Hasan, Karachi (PK); Hafiz Imtiaz Ahmed, Karachi (PK)

(21) Appl. No.: 17/127,604

(22) Filed: Dec. 18, 2020

(30) Foreign Application Priority Data
Nov. 5, 2020 (PK) 759/2020

(51) Int. Cl. A61B 10/00

(52) U.S. Cl. CPC A61B 10/0051; A61B 2010/009

(57) ABSTRACT

A swab is described that includes a handle, a connecting rod including a ring groove, and a head having a shape of at least one of flagella or pilus. The head includes one or more channels configured to collect sample (e.g., specimen) from an anatomical part of a patient. The one or more channels can store the sample while additional sample is being collected. The head has an end that is convex. The handle includes a circular disc to hold the swab. The connecting rod is configured to be broken at the ring groove when pressure is applied to bend the swab. The handle, the connecting rod, and the head form/are a single integrated structure and are inseparable without breaking the single integrated structure. The anatomical part can be a nose or a throat. Related apparatuses, systems, techniques, methods and articles are also within the scope of this disclosure.

### RELATED APPLICATION

This disclosure claims priority to Pakistan Patent Application No. 759/2020, entitled “Swab” and filed on Nov. 5, 2020, the entire contents of which is incorporated herein by reference.

### TECHNICAL FIELD

This disclosure generally relates to swabs used to collect biological samples from an anatomical part (e.g., nose or throat) of a patient.

### BACKGROUND

Swabs are often used for collecting biological samples from various anatomical parts—e.g., back of the nose or throat—of a patient. The swabs containing the collected samples are often immersed in a culture media in a test tube, vial, culture dish, or culture bottle immediately after collection to preserve the collected samples for storage or for transportation to a clinical laboratory. Clinicians or other professionals at the clinical laboratory then analyze such collected samples to detect organisms or other clinical markers. Presence of organisms or other markers in the samples can indicate disease specific to those organisms or other markers.

### SUMMARY

In one aspect, a swab is described that includes a handle, a connecting rod including a ring groove, and a head having a shape of at least one of flagella or pilus. The head includes one or more channels configured to collect a sample (which can also be referred to as specimen) from an anatomical part of a patient. In some implementations consistent with this aspect, one or more of the following can be additionally implemented either individually or in any feasible combination. The one or more channels can store the sample while additional sample is being collected. The head has an end that is convex. The head has a shape of a dome. The handle includes a disc (e.g., circular disc) to hold the swab. The connecting rod is configured to be broken at the ring groove when pressure is applied to bend the swab. The handle, the connecting rod, and the head form/are a single integrated structure and are inseparable without breaking the single integrated structure. The anatomical part can be a nose or a throat.

In another aspect, a method is described that includes: holding a disc (e.g., circular disc) of a swab; inserting the swab into an anatomical part of a patient; and rotating the swab when the swab touches a portion of the anatomical part to collect a sample. Such steps can be performed by, for example, a clinician. In some implementations consistent with this aspect, one or more of the following can be additionally implemented either individually or in any feasible combination. The method can further include: taking out the swab; placing the swab with the collected sample into a collection apparatus; breaking a portion of the swab that does not fit into the collection apparatus by applying pressure at a ring groove within the swab; and sealing the collection apparatus. The swab can include a handle, a connecting rod including a ring groove, and a head having a shape of at least one of flagella or pilus. The head can include one or more channels configured to collect sample from an anatomical part of a patient. The one or more channels can store the sample while additional sample is being collected. The head has an end that is convex. The convex head has a shape of a dome. The handle includes a disc (e.g., circular disc) to hold the swab. The connecting rod is configured to be broken at the ring groove when pressure is applied to bend the swab. The handle, the connecting rod, and the head form/are a single integrated structure and are inseparable without breaking the single integrated structure. The anatomical part can be a nose or a throat.

The details of one or more variations of the subject matter described herein are set forth in the accompanying drawings and the description below. Other features and advantages of the subject matter described herein will be apparent from the description and drawings, and from the claims.

### BRIEF DESCRIPTION OF DRAWINGS

FIG. 1 illustrates an example of a swab used in the detection of respiratory tract infections in a patient, in accordance with some implementations described herein.

FIG. 2 illustrates another example of a swab used in the detection of respiratory tract infections in a patient, in accordance with some implementations described herein.

FIG. 3 illustrates yet another example of a swab used in the detection of respiratory tract infections in a patient, in accordance with some implementations described herein.

FIGS. 4-6 illustrate a unique structure of a swab, in accordance with some implementations described herein.

FIG. 7 illustrates a sampling process, in accordance with some implementations described herein.

Like reference symbols in the various drawings indicate like elements.

### DETAILED DESCRIPTION

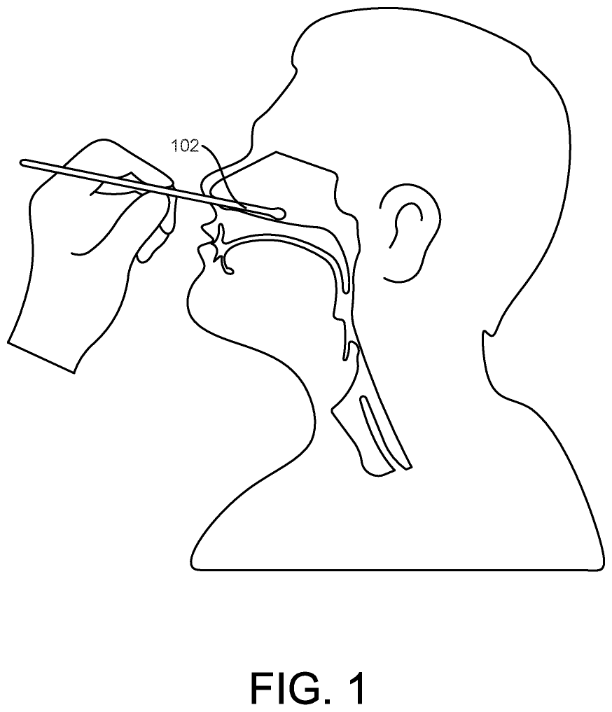

FIG. 1 illustrates an example of a swab 102 used in the detection of respiratory tract infections in a patient. The swab 102 in the shown example is a nasal swab that collects biological samples (which can also be referred to as specimens) from an anatomical part—e.g., nose—of the patient. Various bacterial parasites and anaerobic bacteria accumulate in the nasal region, at least because breath of the patient passes through that region. Some examples of such bacterial parasites include catarrhalis, Staphylococcus, Streptococcus, diphtheria-like bacteria, Klebsiella pneumonia, E. coli, and/or the like. Some examples of anaerobic bacteria include pneumococcus, Bacteroides and/or yeast-like bacteria. Virus can also accumulate in the pharynx 204 because the flu is transmitted through the respiratory tract. Clinicians often use the swab 102 to remove the specimen, which can be in the form of fluid, from the nasal region. To extract the specimen, the swab 102 may, in some implementations, be rotated a preset number of times (e.g., 5 times, or ×5) and inserted in for a preset distance (e.g., 2 inches) for most efficacy. The swab 102 containing such specimen is immersed in a culture media in a test tube, vial, culture dish, or culture bottle immediately after collection to preserve the collected fluid for storage or for transportation to a clinical laboratory. Clinicians, or other professionals at the clinical laboratory, then analyze such collected specimen to detect organisms or other clinical markers. Presence of organisms or other markers in the sample can indicate diseases such as whooping cough, diphtheria, influenza, suppurative tonsillitis, acute pharyngitis, diseases caused by the coronavirus family of viruses—e.g., severe acute respiratory syndrome (SARS), Middle East respiratory syndrome (MERS), and COVID-19, and/or the like—as well as other oral diseases, and/or the like.

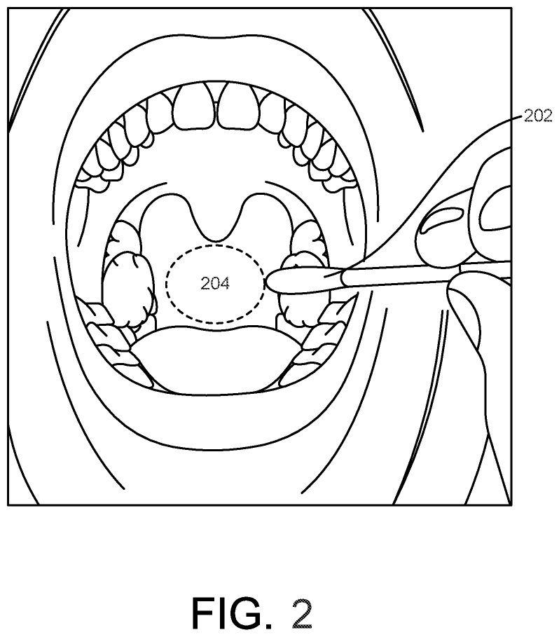

FIG. 2 illustrates another example of a swab 202 used in the detection of respiratory tract infections in a patient. The swab 202 in the shown example is a throat swab that collects biological samples (which can also be referred to as specimens) from an anatomical part—e.g., pharynx 204—of the patient. Various bacterial parasites and anaerobic bacteria accumulate in the pharynx because both the breath and food pass through the pharynx. Some examples of such bacterial parasites include catarrhalis, Staphylococcus, Streptococcus, diphtheria-like bacteria, Klebsiella pneumonia, E. coli, and/or the like. Some examples of anaerobic bacteria include pneumococcus, Bacteroides and/or yeast-like bacteria. Virus can also accumulate in the pharynx 204 because the flu is transmitted through the respiratory tract. Clinicians often use the swab 202 to remove the fluid from the pharyngeal isthmus, which is the passage posterior to the soft palate by which the nasopharynx and oropharynx communicate (i.e., the junction of nasopharynx and oropharynx). The swab 202 containing such fluid is immersed in a culture media in a test tube, vial, culture dish, or culture bottle immediately after collection to preserve the collected fluid for storage or for transportation to a clinical laboratory. Clinicians, or other professionals at the clinical laboratory, then analyze such collected fluid to detect organisms or other clinical markers. Presence of organisms or other markers in the sample can indicate diseases such as whooping cough, diphtheria, influenza, suppurative tonsillitis, acute pharyngitis, diseases caused by the coronavirus family of viruses—e.g., severe acute respiratory syndrome (SARS), Middle East respiratory syndrome (MERS), and COVID-19, and/or the like—as well as other oral diseases, and/or the like.

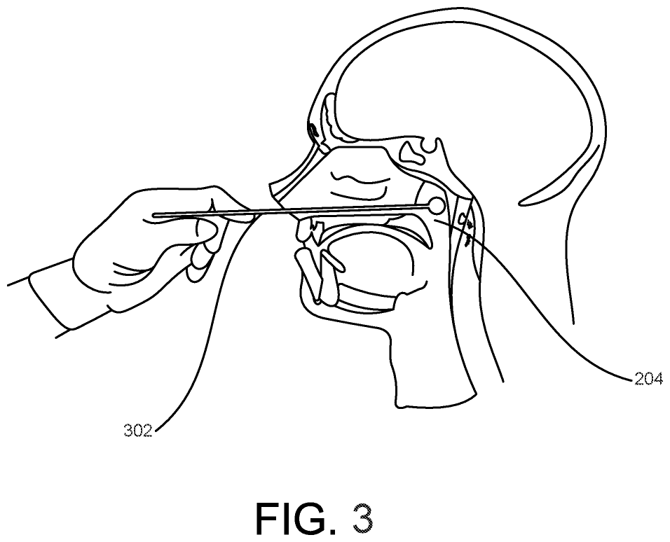

FIG. 3 illustrates another example of a swab 302 used in the detection of respiratory tract infections in a patient. The swab 302 in the shown example is a nasopharyngeal swab that collects biological samples in the form of nasal secretions from the pharynx 204 of the patient. To collect the sample, the swab 302 is inserted in the nostril and gently moved forward into the nasopharynx, a region of the pharynx 204 that covers the roof of the mouth. The swab 302 is then rotated for a specified period time to collect secretions. Subsequently, the swab 302 containing the secretions is immersed in a culture media in a test tube, vial, culture dish, or culture bottle immediately after collection to preserve the collected secretions for storage or for transportation to a clinical laboratory. Clinicians or other professionals at the clinical laboratory then analyze such collected secretions to detect organisms or other clinical markers. Presence of organisms or other markers in the sample can indicate diseases such as whooping cough, diphtheria, influenza, suppurative tonsillitis, acute pharyngitis, diseases caused by the coronavirus family of viruses—e.g., SARS, MERS, and COVID-19, and/or the like—as well as other oral diseases, and/or the like.

Conventional designs of swabs 102, 202, and 302 have various disadvantages. For example, traditional swabs are made up of synthetic or organic material (e.g., flocked tapered swab, or a flocked or spun polyester swab), include a cylindrical rod containing a collection or sampling end, which has a sampling head composed of filaments. Once the sample is collected from the patient, the virus gets trapped inside the fiber structure of the swab and is difficult to release. Thus, such swabs have a small release/collection ratio, and therefore the sample collection effect is laborious, time consuming, and ineffective.

Moreover, hydrophilic short fibers—such as cotton and rayon—have high fluid absorption because they are hydrophilic, and thus the amount of released samples is small. Additionally, sterilization of such traditional swabs is difficult because dipping the cotton swab in ethanol makes the sampling head wet for a long time.

In addition, for the nasal cavity, due to its sensitive skin, when the traditional swab is inserted into the nasal cavity for sampling, it is easy to bruise the nasal cavity and cause bleeding. Similarly, scraping the pharynx 204 can, at times, be harsh on the patient, and cause a gag reflex and/or coughing by the patient, which may force the clinician to delay the sample collection procedure until the nasal or pharyngeal cavity has recovered. Similarly, the sampling head of a conventional swab typically blocks the nasal cavity, and thus it is usually difficult to determine bleeding in the nasal cavity, in which case continued sampling process with the traditional swab further aggravates the bleeding.

Furthermore, some conventional swabs have sampling heads having a large circumference. Such large circumference can cause accidental trauma or bruising of the nasal cavity of the patient.

Additionally, some conventional swabs are calcium alginate swabs or swabs with wooden shafts. Such swabs are disadvantageous because they may contain substances that inactivate some viruses and inhibit polymerase chain reaction (PCR) testing.

Furthermore, table adherence of the fiber wrapped around the end or tip of the rod (i.e., stick) of conventional swabs is generally achieved by gluing. However, gluing can be disadvantageous because the glue can contaminate the collected sample, the glue can loosen, and/or the fibers can unwrap.

Further, fibers in the sampling heads of traditional swabs can be harmful, when exposed to a heated alkaline solvent, to the sensitive skin of the nose of the patient.

Moreover, when cotton is used on an end of a traditional swab, the patient may possibly be prone to biting a cotton swab, which can result in the cotton swab getting stuck in the throat of the patient or swallowed by the patient, which can not only create complications for the patient but also render the sample collection process ineffective.

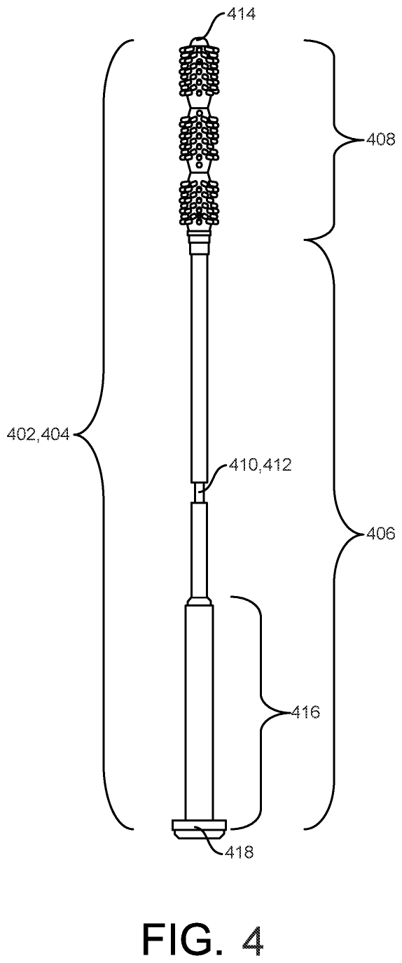

FIG. 4 illustrates a design 402 of a swab 102, 202, or 302 that addresses the deficiencies, including the above-noted disadvantages, of the conventional designs of the swab. A swab with design 402 is also referred to as swab 402 herein. The swab 402 can be used to efficiently collect sample from either the nose or the throat of a patient. The design of the swab 402 can prevent any injuries to the anatomical parts (e.g., interior of nose or throat) or an ineffective collection mechanism even when the clinician operates without reasonable care.

The swab 402 includes a connecting rod 406 and a sampling head 408 (which can also simply be referred to as a head). The connecting rod 406 has a ring groove 410, which serves as a mark of a break point 412, which is a point at which the swab 402 can be broken by a clinician after the clinician takes out the swab 402 from the sampling site (e.g., pharynx) and puts the swab 402 into, for example, a test tube. The break point 412 is designed to be located at a point such that the broken part of the swab 402 that holds the collected sample fits into the test tube. In some implementations, the location of the ring groove 410 (i.e., location of the break point 412) may be varied during the manufacturing process based on the size of the test tube to be used. In a few implementations, different swabs 402 may have different locations of the ring groove 410 (i.e., locations of the break point 412) to ensure compatibility with respective lengths of various test tubes.

The thin structure of the ring groove 410 advantageously makes it convenient for a clinician to break the swab 402 after the sample has been collected. The amount of force required to break the swab 402 at the break point 412 is 500 Newtons or more. In other implementations, the amount of force required to break the swab 402 at the break point 412 may be 1000 Newtons or more. In a few implementations, the amount of force required to break the swab 402 at the break point 412 may be 2000 Newtons or more. The amount of force required to break the swab 402 in this manner can be varied during the manufacturing process by altering the type of material used to form the swab 402, length of the ring groove 410 along the axis of that ring groove 410, thickness/diameter/circumference of the rung groove 410, and/or the like.

After the clinician breaks the swab 402 from the break point 412, the clinician may discard (e.g., throw) the other part of the swab 402 that includes the handle. Once broken, the clinician can insert the part of the swab 402 with the collected sample into a test tube, and cover the test tube before transferring the test tube to a laboratory for clinical analysis of the sample.

The connecting rod 406 includes a handle 416, one end of which is a disc 418. In some examples, the disc 418 can be circular and/or flat. The disc 418 is designed to allow a user (e.g., clinician) to comfortably and firmly hold the swab 402. The clinician may conveniently hold the swab 402 by placing his or her fingers around the disc 418.

The sampling head 408 of the swab 402 has a penetration head 414, which has a convex shape. The convex shape of the penetration head 414 comforts the patient when the swab 402 is inserted into the nose or throat of the patient, and thus allows the surface area of contact between the swab 402 and the sampling site (e.g., pharynx of the patient) to be maximized so as to allow effective collection of the sample.

The sampling head 408 does not have a filament-wound structure (e.g., cotton) like a traditional sampling swab, and thus the swab 402 makes it easy (e.g., does not make it difficult) to release the virus sample after the virus sticks to the sampling head 418.

The sampling head 408 has a flagella-like or pilus-like shape (as noted above). In the shown example, the sampling head 408 has several appendages (i.e., protrusions) of the flagella-like or pilus-like shape along the surface of the sampling head 408. For reference, flagella are long thin appendages, one end of each of which is free and the other end of each of which is attached to a biological cell; and pili are short, thick straight hair-like surface appendages, such as those found in gram negative bacteria. Along any circumference (which is perpendicular to the length) of the sampling head 408 that has many appendages, there are 8-12 appendages. The sampling head 408 has 8-12 appendages across several cross-sections perpendicular to the axis of the swab 402, as shown. While 8-12 appendages are described in each cross-section, in some implementations the number of appendages per cross section can be 4-20. Additionally, the number of cross-sections at the boundary of which the appendages are placed can be varied from those shown to any number between 2 and 50.

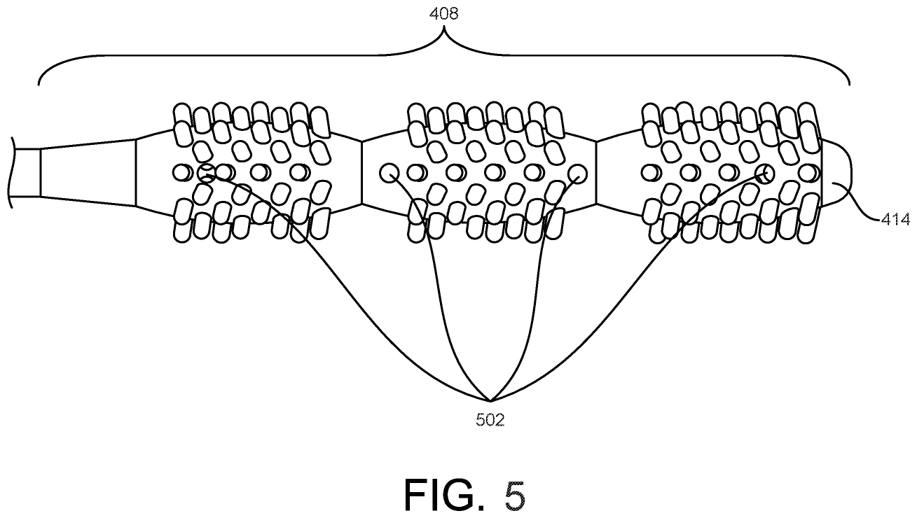

When the swab 402 is rotated by a clinician during the sample collection process, the head 408 rotates and the appendages induce the sample (e.g., nasal secretions) into the channels/holes 502 (as shown in FIG. 5), from which the sample enters the swab 402. The appendages induce the sample into the channels 502 as follows. When the swab 402 is being moved (e.g., rotated inside the anatomical area being tested, or moved in and out of such anatomical area), the anatomical area being tested is brushed by the appendages. The inside of the swab 402 is hollow (or largely hollow, such as 70% or more hollow, in some implementations); thus, when the anatomical area being tested is brushed by the appendages, the movement of the swab 402 helps in or ensures creation of an airflow toward the channels 502. Such airflow creates a suction force, which induces the sample (e.g., fluid sample) into the holes 502. The term induction can also be referred to as: any of attracting, taking up, sweeping up, scooping, grabbing up, and/or the like; and/or facilitating any of attracting, taking up, sweeping up, scooping, grabbing up, and/or the like. The channels 502 can also be referred to as holes, pathways, pipes, tubes, and/or the like in various implementations.

The appendages (i.e., protrusions) also enhance/increase the surface area of the swab 402 that is in contact with the anatomical area being sampled, as compared to a swab without appendages or lesser number of appendages. The enhanced/increased surface area makes the collecting of the sample more efficient (e.g., quicker), as more sample can be collected in lesser time, as compared to collection by a swab without appendages or lesser number of appendages.

Additionally, because the base of the sampling head 408 has a larger circumference than most of the remaining part of the swab 402, more appendages can be present (e.g., designed before manufacturing the swab 402) on the sampling head 408, and thus the induction of the sample is maximized and/or optimized.

Each appendage (i.e., protrusion) on the swab head 402 is at an angle of 15 degrees from a tangent at the underlying point (i.e., point from which the appendage emanates) on the base of the sampling head 408. As the base of the sampling head 408 has a curvature (as shown in FIGS. 4 and 5), the angle of 15 degrees may be measured using different tangents for different appendages. Such 15 degree inclination of the appendages allow a smoother (e.g. gentler) interaction between the sampling head 408 with appendages and the surface of the anatomical part being sampled. Such gentler interaction prevents the patient from being injured by the movement of the swab 402 within the anatomical part. Such gentler interaction also allows an easy and quick pulling out of the swab 402 from the anatomical part after the sample has been collected. While each appendage (i.e., protrusion) is described as at an angle of 15 degrees from a tangent at the underlying point on the base of the sampling head 408, in other implementations the angle can be any value between 10 degrees and 20 degrees. In a few implementations, such angle can be any value between 5 degrees and 30 degrees. In some implementations, only some (e.g., 50%, 70%, 80%, 90%, or the like, in various implementations)—rather than all—appendages may have such angle between any particular appendage and a respective tangent.

The sampling channel/hole 502 is hollow. Such hollowness facilitates storing the sample (e.g., nasal fluid) inside the channel 502. The channel 502 can have a large volume, which allows the swab to extract and collect a large volume of sample (e.g., more volume than conventional swabs). The swab 402 has a higher release/collection ratio when sampling, as compared with conventional swabs, and thus collection of samples using the swab 402 is more effective. It was experimentally determined that the traditional filament wound swab has a 30% release capacity (i.e., ratio of the released amount of sample to the collected amount of sample) for the collected virus samples, and the swab 402 provides a 90% release capacity for the collected microorganisms.

The sampling head 408 has a small diameter as compared to traditional swabs. The small diameter of the sampling head 408 can prevent the sampling head 408 from damaging the epithelium of the internal tissues of the patient. The penetration head 414 of the swab 402 can be dome-shaped. The dome-shaped penetration head 414 minimizes the chance of an injury to the patient, as opposed to sharp ends on some conventional designs of the swab that can cause injury to (e.g., bleeding in the nasal cavity of) the patient. The smaller diameter of the sampling head 408 and the dome-like shape of the penetration head 414 minimizes the risk of injury when the swab 402 is accidently pushed against the sampling site (e.g., pharynx) on the patient.

The number and location of channels 502 (as shown in FIG. 5) within the sampling head 408 allow a quick (e.g., rapid) absorption of the quantity of sample to be collected and tested. For example, when the swab 402 is rotated by a clinician during the sample collection process, the head 408 rotates and the appendages induce the sample (e.g., nasal secretions) into the channels/holes 502, from which the sample enters the swab 402. The appendages induce the sample into the channels 502 as follows. When the swab 402 is being moved (e.g., rotated inside the anatomical area being tested, or moved in and out of such anatomical area), the anatomical area being tested is brushed by the appendages. The inside of the swab 402 is hollow (or largely hollow, such as 70% or more hollow, in some implementations); thus, when the anatomical area being tested is brushed by the appendages, the movement of the swab 402 helps in or ensures creation of an airflow toward the channels 502. Such airflow creates a suction force, which induces the sample (e.g., fluid sample) into the holes 502. The number and location of channels 502, as shown closely in FIG. 5, allows the swab 402 to collect a large (e.g., maximum) amount of sample in less (e.g., least) time. In some other implementations, the number and/or location of the channels 502 can be varied. For example, in some implementations, the number of channels 502 can be varied to any number between 1 and 20, and the locations of those channels 502 is evenly (or almost evenly, in some implementations) distributed on the surface of the base of the sampling head 408.

The swab 402 has a slim body because of a thin diameter. The slim body enables the swab 402 to pass easily through the nostril and travel parallel to the palate. Additionally, the slim body protects the swab 402 from being contaminated by the mucous membrane and mucus before and after sampling, which increases the sampling accuracy and decreases the number of inaccurate test results.

When using the swab 402, the clinician can first hold the disc 418 and gently insert the swab 402 into the nasal cavity; then gently rotate the swab 402 three to five times to collect the sample; then take out the swab 402 slowly; then put the extracted sample (e.g., virus sample) into a collection tube (e.g., test tube); then break the excess portion of the swab 402 (i.e., the portion of the swab 402 that does not fit into the collection tube) from the break point; then store the sampling head 408 in the collection tube; and then seal the collection tube to complete the sampling process.

The swab 402 allows convenient collection of sample, maintenance of hygiene during the use or storage of the swab 402, safety during the collection process, and convenient disassembly after collection. The swab 402 can be manufactured by implementing various manufacturing methods, including 3D printing, which can allow uniformity of the design and accordingly uniformity in the process of collection of sample. The swab 402 is disposable, and can be manufactured at a low budget.

The swab 402 can be sterilized in an easy manner. For example, the swab 402 can be dipped in ethanol without any harmful effects being caused by such dipping in conventional swabs (e.g., dipping a traditional cotton swab in ethanol makes the sampling head wet for a long time).

The swab 402 is made up of plastic that cannot be broken easily. Some examples of the plastic that can be used include polyethylene, polypropylene, polycarbonate, polyoxymethylene, acrylonitrile butadiene styrene (ABS), and/or the like. Because such plastic cannot be broken easily, use of such plastic to form the swab 402 prevents the swab 402 from breaking or losing structural shape if the patient accidentally (or even intentionally) bites, or applies force on, the swab 402 during the sample collection process (e.g., during the process when the clinician removes the fluid sample from the pharyngeal isthmus with the swab 402).

Further, use of plastic, instead of synthetic fiber, for the sampling head 408 eliminates the risk of fibers attaching to the inner lining of the nasal track of the patient when the swab 402 is rubbed and/or rolled during sample collection, thereby ensuring comfort and convenience of the patient. The plastic used for making the swab is non-toxic and made from a biocompatible resin; therefore, leaving the swab 402 in place for several seconds to absorb secretions, which can be filled up in the sampling channels/holes 502, do not create discomfort, or strike a reflex response of sneezing, in the patient.

Additionally, the use of plastic for the shaft of the swab 402 provides the swab 402 more flexibility than wooden or wire shafts in conventional swabs, and as a result of such flexibility the sample extraction procedure is smoother for both the clinician and the patient. Further, because the sampling head 408 is made of plastic, the sample (e.g., virus sample) does not get adsorbed within the material forming the sampling head 408 and rather sticks to the surface of the sampling head 408, and can be easily released.

While the swab 402 is described as being made of plastic, in some implementations the swab 402, or some portions of the swab 402, can be made of any other material. For example, in particular implementations, the swab 402, or some portions thereof, can be made of any organic compound. The organic compound may be natural, synthetic, or semi-synthetic. The material used to form the swab 402 is environment-friendly, nontoxic, tasteless, autoclavable (i.e., able to withstand the action of an autoclave) and/or biocompatible.

The disc 418 allows the clinician a firm grip to the swab 402, and hence the swab 402 cannot slide when sampling. Uniform diameter is maintained all around the different portions of the swab 402 such as the head, break point and handle along with a uniform circular cross section. The swab 402 allows stable sample collection volumes, high release efficiency of collected samples, and does not induce pain or discomfort in the subject. The swab 402 can produce accurate results for diagnostic purposes. Additionally, the swab 402 is easy to mass-produce and is economical. The swab 402 can have a low selling price. The swab 402 can be produced/manufactured in bulk, thereby making the swab 402 attractive in situations when mass testing needs to be performed, such as during a pandemic.

The swab 402 can be resistant to higher temperatures and does not deform when exposed to sunlight or higher temperatures, as opposed to traditional swabs that require a particular temperature range for storage. Thus, the swab 402 has a long shelf life (e.g., shelf life ranging from a few months to many years, depending on the place of storage of the swab 402).

The entire swab 402 is a single continuous integrated structure 404. Because the swab 402 is a single integrated unit, there is no possibility of any part of the swab 402 remaining in the tract during collection of the sample (unlike in traditional swabs where there is a risk that the fibers can be left in the anatomical parts of the patient when the sample is being collected). The single integrated structure 404 obviates the need for glue (as required in some traditional swabs to combine/couple different components of those traditional swabs), which may be required in conventional swabs where fiber may need to be glued around the end or tip of the shaft of a conventional swab. Thus the design feature of there being a single continuous integrated structure obviates the disadvantages of glue, such as contamination of the collected sample with the glue. Furthermore, the single integrated structure prevents different parts (e.g., connecting rod 406 and sampling head 408) of the swab 402 from separating from other parts or falling off during the sampling process.

When using the swab 402, a user (e.g., clinician) can first hold the swab 402 from the disc 418. The disc 418 can provide a firm grip to the clinician even when the clinician is wearing clinical or surgical gloves (e.g., latex gloves, or latex free gloves), thereby avoiding clinical accidents with the swab. The disc 418 can be circular, as noted above. In some other implementations, the disc 418 can have another shape that may provide an even firmer grip than a circular disc. Such other shape can be a polygon with any number of sides, or any irregular shape that allows the holding of the disc, and thus the holding of the swab 402, comfortable.

The clinician can then gently insert the swab 402 into the nasal cavity and rotate the rod 3-5 times. The flagella-like or pilus-like structure of the sampling head 408 brushes against the nasal cavity and attaches the fluid along the appendages (i.e., projections) of that structure. The sampling channels/holes 502 can accumulate the excess sample fluid present in the nasal cavity and retain the fluid while the clinician takes out the swab 402 slowly and then puts the extracted sample into the collection tube, breaks the excess rod from the break point 412, stores the sampling head 408 in the collection tube, and seals the collection tube to complete the sampling process.

The swab 402 is transparent or semi-transparent, which informs the patient to some extent as to how the swab 402 works, thereby enhancing comfort-especially psychological comfort-when the sample is being extracted from the patient. While the swab is described as transparent or semi-transparent, in other implementations, some or all of the swab 402 may be opaque.

FIG. 5 illustrates a closer view of the head 408 of the swab 402. This view shows, among other things, the appendages (i.e., protrusions) on the head 408 and the channels/holes 502 in the head 408.

The sampling head 408 of the swab 402 has a penetration head 414, which has a convex shape. The convex shape of the penetration head 414 comforts the patient when the swab 402 is inserted into the nose or throat of the patient, and thus allows the surface area of contact between the swab 402 and the sampling site (e.g., pharynx of the patient) to be maximized so as to allow effective collection of the sample.

The sampling head 408 does not have a filament-wound structure (e.g., cotton) like a traditional sampling swab, and thus the swab 402 makes it easy (e.g., does not make it difficult) to release the virus sample after the virus sticks to the sampling head 418.

The sampling head 408 has a flagella-like or pilus-like shape (as noted above). In the shown example, the sampling head 408 has several appendages (i.e., protrusions) of the flagella-like or pilus-like shape along the surface of the sampling head 408. For reference, flagella are long thin appendages, one end of each of which is free and the other end of each of which is attached to a biological cell; and pili are short, thick straight hair-like surface appendages, such as those found in gram negative bacteria. Along any circumference (which is perpendicular to the length) of the sampling head 408 that has many appendages, there are 8-12 appendages. The sampling head 408 has 8-12 appendages across several cross-sections perpendicular to the axis of the swab 402, as shown. While 8-12 appendages are described in each cross-section, in some implementations the number of appendages per cross section can be 4-20. Additionally, the number of cross-sections at the boundary of which the appendages are placed can be varied from those shown to any number between 2 and 50.

When the swab 402 is rotated by a clinician during the sample collection process, the head 408 rotates and the appendages induce the sample (e.g., nasal secretions) into the channels/holes 502, from which the sample enters the swab 402. Because the sampling head 408 has a larger circumference than most of the remaining part of the swab 402, more appendages can be present, and thus induction of the sample is maximized and/or optimized.

The structural orientation of the sampling channel/hole 502 helps to collect the sample more efficiently when rotating inside the nasal cavity, and easily pull back from the anatomical part (e.g., nasal cavity) at the end of sampling. The hollow sampling channel/hole 502 can store the sample (e.g., nasal fluid) inside, which means a large volume of sample can be extracted and collected (e.g., more volume than conventional swabs). The swab 402 has a higher release/collection ratio when sampling, as compared with conventional swabs, and thus collection of samples using the swab 402 is more effective. It was experimentally determined that the traditional filament wound swab has a 30% release capacity (i.e., ratio of the released amount of sample to the collected amount of sample) for the collected virus samples, and the swab 402 provides a 90% release capacity for the collected microorganisms.

The sampling head 408 has a small diameter as compared to traditional swabs. The small diameter of the sampling head 408 can prevent the sampling head 408 from damaging the epithelium of the internal tissues of the patient. The penetration head 414 of the swab 402 can be dome-shaped. The dome-shaped penetration head 414 minimizes the chance of an injury to the patient, as opposed to sharp ends on some conventional designs of the swab that can cause injury to (e.g., bleeding in the nasal cavity of) the patient. The smaller diameter of the sampling head 408 and the dome-like shape of the penetration head 414 minimizes the risk of injury when the swab 402 is accidently pushed against the sampling site (e.g. pharynx) on the patient.

The number and location of channels 502 within the sampling head 408 allows a rapid absorption of the quantity of sample to be collected and tested.

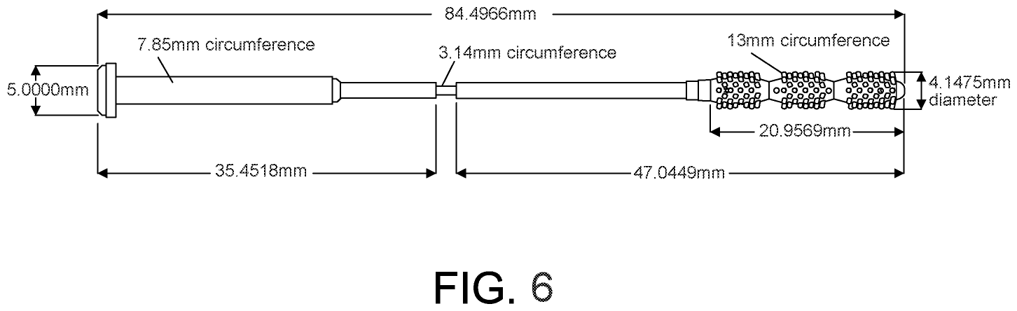

FIG. 6 illustrates dimensions of one example of the swab 402. The total length of the swab 402 in the shown implementation is 84.5 mm. In some implementations, the total length of the swab 402 is any value between 80 mm and 90 mm. In a few implementations, the total length of the swab 402 is any value between 60 mm and 110 mm.

The sampling head 408 has the total length of 20.95 mm, width (e.g., diameter of cross-section that is perpendicular) of 4.14 mm, and the circumference of 13 mm. In some implementations, the total length of the sampling head 408 can be any value between 17 mm and 23 mm. In certain implementations, the total length of the sampling head 408 can be any value between 15 mm and 25 mm. In some implementations, the width of the sampling head 408 can be any value between 3 mm and 5 mm. In few implementations, the width of the sampling head 408 can be any value between 2 mm and 6 mm. In some implementations, the circumference of the sampling head 408 can be any value between 11 mm and 13 mm. In particular implementations, the circumference of the sampling head 408 can be any value between 9 mm and 15 mm.

The width (i.e., diameter) of the circular disc 418 is 5.0 mm. In some implementations, the width (i.e., diameter) of the circular disc 418 can be any value between 4 mm and 6 mm. In certain implementations, the width (i.e., diameter) of the circular disc 418 can be any value between 3 mm and 7 mm.

The other portion of the handle 416 (i.e., portion other than where the disc 418 is located) has a circumference of 7.85 mm. In some implementations, the circumference of that other portion of the handle 416 can be any value between 6.5 mm and 8.8 mm. In a few implementations, the circumference of that other portion of the handle 416 can be any value between 5 mm and 10 mm.

The break point 412 is 1.99 mm long along the axis of the swab 402, and has a circumference of 3.14 mm. In some implementations, the length of the breakpoint 412 along the axis of the swab 402 is any value between 1.5 mm and 2.5 mm. In a few implementations, the length of the breakpoint 412 along the axis of the swab 402 is any value between 1 mm and 3 mm.

Other dimensions, if not noted above, are shown in the drawing. In some other implementations of the swab 402, any of the shown dimensions can be varied—reduced or increased—by up to 10%. In a few implementations of the swab 402, any of the shown dimensions can be varied—reduced or increased—by up to 25%. In particular implementations of the swab 402, any of the shown dimensions can be varied—reduced or increased—by up to 50%. In certain implementations of the swab 402, any of the shown dimensions can be varied—reduced or increased—by any amount that is feasible such that the desired benefits, as discussed herein, of the swab 402 are attainable.

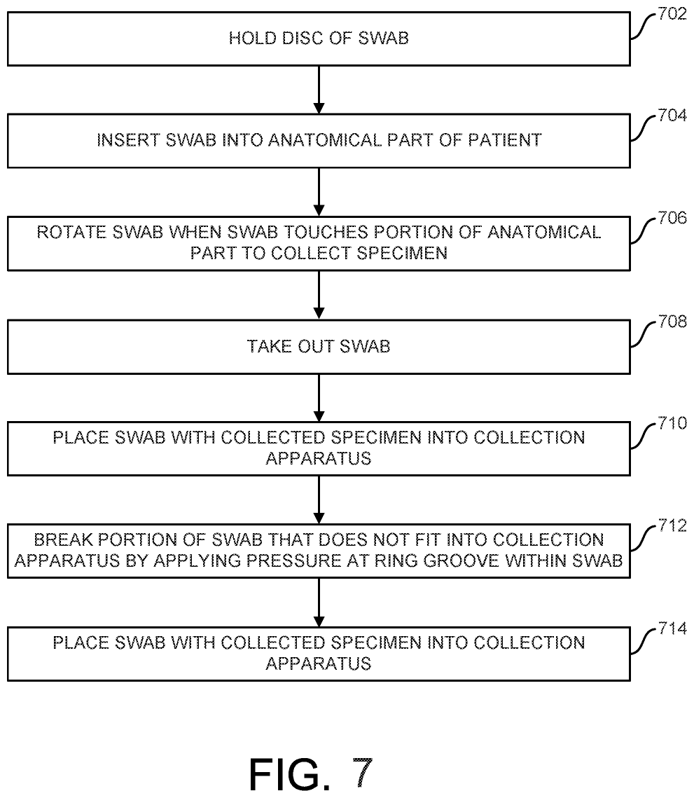

FIG. 7 illustrates a sampling process to effectively collect and store a sample by using the swab 402. The process can be performed by a user, which is generally a clinician. While such process is described as being performed by the clinician, in some implementations the user performing the process can be the patient or any other user. In some implementations, different steps of the process can be performed by different users, such that any one or more steps can be performed by the clinician while other steps are performed by the patient. For ease of reference, the steps below are generally described as being performed by a user.

The user can hold, at 702, the disc 418 of the swab 402. The user can insert, at 704, the swab 402 into an anatomical part (e.g., pharynx 204) of a patient. The user can rotate, at 706, the swab 402 when the swab 402 touches a portion of the anatomical part (e.g., pharynx 204) to collect a sample. The user can take out, at 708, the swab 402 from the anatomical part (e.g., by taking out the swab 402 from the nose or throat of the patient). The user can place, at 710, the swab 402 with the collected sample into a collection apparatus, such as a test tube. The user can break, at 712, a portion of the swab 402 that does not fit into the collection apparatus by applying pressure at a ring groove 410 within the swab 402. The user can seal, at 714, the collection apparatus.

The collection apparatus can be transported to a laboratory. Clinical or other professionals at the clinical laboratory can then analyze the sample to detect organisms or other clinical markers in the sample. Presence of organisms or other markers in the sample can indicate one or more diseases such as whooping cough, diphtheria, influenza, suppurative tonsillitis, acute pharyngitis, diseases caused by the coronavirus family of viruses—e.g., SARS, MERS, and COVID-19, and/or the like—as well as other oral diseases, and/or the like.

The term sample, as used herein, can include one or more of secretions, tumors, biological tissues, extracted nucleic acids such as one or more of deoxyribonucleic acid or ribonucleic acid, urine, plasma, serum, blood cells, and/or the like.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some examples be excised from the combination, and the claimed combination may be directed to a sub-combination or variation of a sub-combination.

Although a few variations have been described in detail above, other modifications are possible. For example, the logic flows described herein may not require the particular order shown, or sequential order, to achieve desirable configurations or results. Other embodiments may be within the scope of the following claims.

1. A swab comprising:
a handle;
a connecting rod comprising a ring groove; and
a head having a shape of at least one of flagella or pilus, wherein the head includes one or more channels configured to collect sample from an anatomical part of a patient.

2. The swab of claim 1, wherein the one or more channels are configured to store the sample while additional sample is being collected.

3. The swab of claim 1, wherein the head has an end that is convex.

4. The swab of claim 3, wherein the head has a shape of a dome.

5. The swab of claim 1, wherein the handle includes a circular disc to hold the swab.

6. The swab of claim 1, wherein the connecting rod is configured to be broken at the ring groove when pressure is applied to bend the swab.

7. The swab of claim 1, wherein the handle, the connecting rod, and the head are a single integrated structure and are inseparable without breaking the single integrated structure.

8. The swab of claim 1, wherein the anatomical part is a nose or a throat.

9. A method comprising:
holding a circular disc of a swab;
inserting the swab into an anatomical part of a patient; and
rotating the swab when the swab touches a portion of the anatomical part to collect a sample.

10. The method of claim 9, further comprising:
taking out the swab;
placing the swab with the collected sample into a collection apparatus;
breaking a portion of the swab that does not fit into the collection apparatus by applying pressure at a ring groove within the swab; and
sealing the collection apparatus.

11. The method of claim 9, wherein the swab comprises:
a handle;
a connecting rod comprising a ring groove; and
a head having a shape of at least one of flagella or pilus, wherein the head includes one or more channels configured to collect the sample from the anatomical part.

12. The method of claim 11, wherein the one or more channels are configured to store the sample while additional sample is being collected.

13. The method of claim 11, wherein the head has an end that is convex.

14. The method of claim 13, wherein the head has a shape of a dome.

15. The method of claim 11, wherein the handle comprises the circular disc to hold the swab.

16. The method of claim 11, wherein the breaking of the portion of the swab comprises:
applying pressure at the ring groove to bend the swab, wherein the application of the pressure breaks the portion of the swab.

17. The method of claim 11, wherein the handle, the connecting rod, and the head are a single integrated structure and are inseparable without breaking the single integrated structure.

18. The method of claim 9, wherein the anatomical part is a nose or a throat.
